(19) United States
(12) Patent Application Publication
(10) Pub. No.: US 2020/0126031 A1
(43) Pub. Date: Apr. 23, 2020

(54) System, Method, and Apparatus for Dispensing and Monitoring Pharmacy Shipments

(71) Applicants: Teresa Whalen, Pittsburgh, PA (US); Palma Constantin, Pittsburgh, PA (US); ARMADA SUPPLY CHAIN SOLUTIONS, LLC, Pittsburgh, PA (US)

(72) Inventors: Teresa WHALEN, Pittsburgh, PA (US); Palma CONSTANTIN, Pittsburgh, PA (US); John BURKE, Pittsburgh, PA (US); James R. CONSTANTIN, Pittsburgh, PA (US); Holly CUNDIEFF, Pittsburgh, PA (US)

(73) Assignee: Armada Supply Chain Solutions, LLC, Pittsburgh, PA (US)

(21) Appl. No.: 16/470,389

(22) PCT Filed: Dec. 19, 2017
(86) PCT No.: PCT/US2017/067291

§ 371 Date: Jun. 17, 2019

Related U.S. Application Data
(60) Provisional application No. 62/436,087, filed on Dec. 19, 2016.

(51) Int. Cl. G06Q 10/08; G16H 20/10; G06F 9/54

(52) U.S. Cl. CPC G06Q 10/0833; G06Q 10/0832; G06F 9/542; G16H 20/10; G06Q 10/087

(57) ABSTRACT

Provided is a computer-implemented method for predictively monitoring a shipment of at least one medication order from a first location to a destination location. The method includes receiving a medication order including medication order data identifying at least one medication for a patient, determining predicted environmental conditions for the shipment of the at least one medication from the first location to the destination location based at least partially on the medication order data, determining an optimized packing configuration of at least one of a cooling pack and insulating material for the at least one medication order based at least partially on the predicted environmental conditions, and generating a visual representation of the optimized packing configuration on a display device. A system and computer-program product are also disclosed.

### CROSS REFERENCE TO RELATED APPLICATION

This application claims priority to U.S. Provisional Patent Application No. 62/436,087, filed Dec. 19, 2016, entitled “System, Method, and Apparatus for Dispensing and Monitoring Pharmacy Shipments,” the entire disclosure of which is hereby incorporated by reference.

### BACKGROUND OF THE INVENTION

### Field of the Invention

This invention relates generally to shipments of perishable items and, in particular, to a system, method, and computer program product for dispensing and monitoring pharmacy shipments.

### Technical Considerations

Medications are often shipped from a pharmacy to a patient. Many such medications are sensitive to degradation or alteration if exposed to conditions outside recommended guidelines. In addition, pharmacies and other distributors in the United States involved in the shipment of such medication orders must comply with the Drug Quality and Security Act of 2013 and the Drug Supply Chain Security Act, the latter of which mandates the exchange of information at the individual package level. The Drug Supply Chain Security Act aims to enable verification of the legitimacy of the drug product identifier down to the package level, enhance detection and notification of illegitimate products in the drug supply chain, facilitate more efficient recalls of drug products, and provide lot level product tracing information following the transaction.

An estimated 30-50% of the specialty pharmacy market requires refrigeration and special handling of medication orders. There is limited existing infrastructure to support cold chain monitoring, especially between a specialty pharmacy and the patient or caregiver. Therapeutic efficacy of these high cost pharmaceutical therapies is dependent on the manufacturer stability guidelines and the associated integrity of the cold chain. Cold chain integrity needs to be ensured throughout the transportation of a medication order from the pharmacy facility to the patient or caregiver.

Thus, a system and method for dispensing and monitoring pharmacy shipments is needed to prevent or reduce sub-standard chain of custody processes, to ensure efficacy of shipped medications by adhering to manufacturer storage and excursion guidelines, and to track lot numbers and expiration dates of shipped medications. There is also a need for a system and method to document, store, track, and provide reporting and real-time machine learning to continuously optimize and monitor the chain of custody for such goods.

### SUMMARY OF THE INVENTION

It is an object of the present invention to provide a system, method, and computer program product for dispensing and monitoring pharmacy shipments and/or other perishable items that overcomes some or all of the deficiencies of the prior art.

According to a non-limiting embodiment of the present invention, provided is a computer-implemented method for predictively monitoring a shipment of at least one medication order from a first location to a destination location, comprising: receiving a medication order comprising medication order data identifying at least one medication for a patient; determining, with at least one processor, predicted environmental conditions for the shipment of the at least one medication from the first location to the destination location based at least partially on the medication order data; determining, with at least one processor, an optimized packing configuration of at least one of a cooling pack and insulating material for the at least one medication order based at least partially on the predicted environmental conditions; and generating a visual representation of the optimized packing configuration on a display device.

According to another non-limiting embodiment of the present invention, provided is a system for predictively monitoring a shipment of at least one medication order from a first location to a destination location, comprising at least one computer including at least one processor, the at least one computer programmed or configured to: receive a medication order comprising medication order data identifying at least one medication for a patient; determine predicted environmental conditions for the shipment of the at least one medication from the first location to the destination location based at least partially on the medication order data; determine an optimized packing configuration of at least one of a cooling pack and insulating material for the at least one medication order based at least partially on the predicted environmental conditions; and generate a visual representation of the optimized packing configuration on a display device.

According to a further non-limiting embodiment of the present invention, provided is a computer program product for predictively monitoring a shipment of at least one medication order from a first location to a destination location, comprising at least one computer-readable medium including program instructions that, when executed by at least one processor of at least one computer, cause the at least one computer to: receive a medication order comprising medication order data identifying at least one medication for a patient; determine predicted environmental conditions for the shipment of the at least one medication from the first location to the destination location based at least partially on the medication order data; determine an optimized packing configuration of at least one of a cooling pack and insulating material for the at least one medication order based at least partially on the predicted environmental conditions; and generate a visual representation of the optimized packing configuration on a display device.

Further preferred and non-limiting embodiments or aspects are set forth in the following numbered clauses.

Clause 1: A computer-implemented method for predictively monitoring a shipment of at least one medication order from a first location to a destination location, comprising: receiving a medication order comprising medication order data identifying at least one medication for a patient; determining, with at least one processor, predicted environmental conditions for the shipment of the at least one medication from the first location to the destination location based at least partially on the medication order data; determining, with at least one processor, an optimized packing configuration of at least one of a cooling pack and insulating material for the at least one medication order based at least partially on the predicted environmental conditions; and generating a visual representation of the optimized packing configuration on a display device.

Clause 2: The computer-implemented method of clause 1, further comprising: collecting environmental condition data during shipment of the at least one medication from the first location to the destination location; and determining whether the shipment of the at least one medication complies with predetermined transportation conditions by comparing the collected environmental condition data to the predetermined transportation conditions.

Clause 3: The computer-implemented method of clauses 1 or 2, wherein the predicted environmental conditions are determined based at least partially on at least one of the following: a thermodynamic property of a packaging material, a geographical coordinate, an environmental condition, a shipping route, a mode of transportation, or any combination thereof.

Clause 4: The computer-implemented method of any of clauses 1-3, wherein the predicted environmental conditions are determined based at least partially on at least one thermodynamic property of a packaging material.

Clause 5: The computer-implemented method of any of clauses 1-4, wherein the predicted environmental conditions are determined based at least partially on a predictive algorithm, and wherein the predictive algorithm is modified based on the collected environmental condition data.

Clause 6: The computer-implemented method of any of clauses 1-5, further comprising generating an alert in response to determining that the shipment of the at least one medication complies or does not comply with the predetermined transportation conditions.

Clause 7: The computer-implemented method of any of clauses 1-6, further comprising transmitting the alert to at least one of the following: the patient, a caregiver, a pharmacy, a physician, or any combination thereof.

Clause 8: The computer-implemented method of any of clauses 1-7, wherein the optimized packing configuration is determined based at least partially on an optimized packing algorithm, and wherein the optimized packing algorithm is modified and/or influenced based on the collected environmental condition data.

Clause 9: The computer-implemented method of any of clauses 1-8, further comprising: generating, with at least one processor, a unique identifier for the medication order; storing or embedding the unique identifier in at least one data source that is affixed to a package including the medication order; and linking, in at least one database, the unique identifier to at least a portion of the medication order.

Clause 10: The computer-implemented method of any of clauses 1-9, further comprising tracking the package during shipment from the first location to the destination location by periodically scanning or reading the at least one data source to retrieve the unique identifier and transmitting, to at least one remote server, shipment data and the unique identifier.

Clause 11: The computer-implemented method of any of clauses 1-10, wherein the medication order data further comprises at least one of a lot number and an expiration date, the method further comprising: determining if the shipment should be recalled during shipment based at least partially on the at least one of the lot number and the expiration date; generating an alert in response to determining that the shipment should be recalled; and transmitting the alert to at least one of the following: the patient, a caregiver, a pharmacy, a physician, or any combination thereof.

Clause 12: The computer-implemented method of any of clauses 1-11, further comprising: collecting shipment data during shipment of the at least one medication from the first location to the destination location; and after the at least one medication has been delivered to the destination location, determining whether the shipment of the at least one medication complies with manufacturer data associated with the at least one medication.

Clause 13: The computer-implemented method of any of clauses 1-12, further comprising generating correlated temperature data based on past shipment data and historical environmental data, the correlated temperature data comprising correlations between ambient outdoor temperature measurements associated with past shipments and historical environmental conditions in at least one shipping lane, wherein the predicted environmental conditions are determined based at least partially on the correlated temperature data.

Clause 14: A system for predictively monitoring a shipment of at least one medication order from a first location to a destination location, comprising at least one computer including at least one processor, the at least one computer programmed or configured to: receive a medication order comprising medication order data identifying at least one medication for a patient; determine predicted environmental conditions for the shipment of the at least one medication from the first location to the destination location based at least partially on the medication order data; determine an optimized packing configuration of at least one of a cooling pack and insulating material for the at least one medication order based at least partially on the predicted environmental conditions; and generate a visual representation of the optimized packing configuration on a display device.

Clause 15: The system of clause 14, wherein the at least one computer is further programmed or configured to: collect environmental condition data during shipment of the at least one medication from the first location to the destination location; and determine whether the shipment of the at least one medication complies with predetermined transportation conditions by comparing the collected environmental condition data to the predetermined transportation conditions.

Clause 16: The system of clauses 14 or 15, wherein the predicted environmental conditions are determined based at least partially on at least one of the following: a thermodynamic property of a packaging material, a geographical coordinate, an environmental condition, a shipping route, a mode of transportation, or any combination thereof.

Clause 17: The system of any of clauses 14-16, wherein the predicted environmental conditions are determined based at least partially on at least one thermodynamic property of a packaging material.

Clause 18: The system of any of clauses 14-17, wherein the predicted environmental conditions are determined based at least partially on a predictive algorithm, and wherein the predictive algorithm is modified based on the collected environmental condition data.

Clause 19: The system of any of clauses 14-18, wherein the at least one computer is further programmed or configured to generate an alert in response to determining that the shipment of the at least one medication complies or does not comply with the predetermined transportation conditions.

Clause 20: The system of any of clauses 14-19, wherein the at least one computer is further programmed or configured to transmit the alert to at least one of the following: the patient, a caregiver, a pharmacy, a physician, or any combination thereof.

Clause 21: The system of any of clauses 14-20, wherein the optimized packing configuration is determined based at least partially on an optimized packing algorithm, and wherein the optimized packing algorithm is modified and/or influenced based on the collected environmental condition data.

Clause 22: The system of any of clauses 14-21, wherein the at least one computer is further programmed or configured to: generate a unique identifier for the medication order; store or embed the unique identifier in at least one data source that is affixed to a package including the medication order; and link, in at least one database, the unique identifier to at least a portion of the medication order.

Clause 23: The system of any of clauses 14-22, wherein the at least one computer is further programmed or configured to track the package during shipment from the first location to the destination location by periodically scanning or reading the at least one data source to retrieve the unique identifier and transmitting, to at least one remote server, shipment data and the unique identifier.

Clause 24: The system of any of clauses 14-23, wherein the medication order data further comprises at least one of a lot number and an expiration date, and wherein the at least one computer is further programmed or configured to: determine if the shipment should be recalled during shipment based at least partially on the at least one of the lot number and the expiration date; generate an alert in response to determining that the shipment should be recalled; and transmit the alert to at least one of the following: the patient, a caregiver, a pharmacy, a physician, or any combination thereof.

Clause 25: The system of any of clauses 14-24, wherein the at least one computer is further programmed or configured to: collect shipment data during shipment of the at least one medication from the first location to the destination location; and after the at least one medication has been delivered to the destination location, determine whether the shipment of the at least one medication complies with manufacturer data associated with the at least one medication.

Clause 26: The system of any of clauses 14-25, wherein the at least one computer is further programmed or configured to generate correlated temperature data based on past shipment data and historical environmental data, the correlated temperature data comprising correlations between ambient outdoor temperature measurements associated with past shipments and historical environmental conditions in at least one shipping lane, wherein the predicted environmental conditions are determined based at least partially on the correlated temperature data.

Clause 27: A computer program product for predictively monitoring a shipment of at least one medication order from a first location to a destination location, comprising at least one computer-readable medium including program instructions that, when executed by at least one processor of at least one computer, cause the at least one computer to: receive a medication order comprising medication order data identifying at least one medication for a patient; determine predicted environmental conditions for the shipment of the at least one medication from the first location to the destination location based at least partially on the medication order data; determine an optimized packing configuration of at least one of a cooling pack and insulating material for the at least one medication order based at least partially on the predicted environmental conditions; and generate a visual representation of the optimized packing configuration on a display device.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a”, “an”, and “the” include plural referents unless the context clearly dictates otherwise.

### BRIEF DESCRIPTION OF THE DRAWINGS

Additional advantages and details of the invention are explained in greater detail below with reference to the exemplary embodiments that are illustrated in the accompanying schematic figures, in which:

FIG. 1A is a schematic diagram for a system for dispensing and monitoring pharmacy shipments according to the principles of the present invention;

FIG. 1B is a schematic diagram for a system for dispensing and monitoring pharmacy shipments according to the principles of the present invention;

FIG. 2 is another schematic diagram for a system for dispensing and monitoring pharmacy shipments according to the principles of the present invention;

FIG. 3 is a further schematic diagram for a system for dispensing and monitoring pharmacy shipments according to the principles of the present invention; and

FIG. 4 is a flow diagram for a method for dispensing and monitoring pharmacy shipments according to the principles of the present invention.

### DESCRIPTION OF THE PREFERRED EMBODIMENTS

For purposes of the description hereinafter, it is to be understood that the invention may assume various alternative variations and step sequences, except where expressly specified to the contrary. It is also to be understood that the specific devices and processes illustrated in the attached drawings, and described in the following specification, are simply exemplary embodiments or aspects of the invention. Hence, specific dimensions and other physical characteristics related to the embodiments or aspects disclosed herein are not to be considered as limiting.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that may be wired and/or wireless in nature. Additionally, two units or components may be in communication with each other even though the data transmitted may be modified, processed, and/or routed between the first and second unit or component. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

As used herein, the terms “environmental data” and “environmental condition data” may refer to data representing one or more environmental condition parameters such as, for example, temperature, humidity, pressure, precipitation amount, and/or any other like parameters for measuring and/or quantifying weather and environmental conditions.

According to a preferred and non-limiting embodiment of the present invention, a system and method for dispensing and monitoring shipments of medication orders is provided. The system and method may be used for packaging, storing, and handling pharmaceuticals during preparation and transportation of a medication order. Non-limiting embodiments of the system and method disclosed herein also allow for lot-level management, drug recall handling, tracking, and tracing of prescription medication orders. These features provide the stakeholders, including the patients, caregivers, pharmacies, insurance companies, and manufacturers, with access to data for clinical decision making. Non-limiting embodiments of the present invention also allow for the stakeholders to ensure patient safety, to identify adulterated medications, and to comply with the Drug Supply Chain Security Act.

In a preferred and non-limiting embodiment, the present invention is used to package and/or monitor a medication order for an individual patient. As used herein, the term “medication order” may refer to one or more pharmaceuticals intended for a particular recipient (e.g., a patient or patient caregiver). This may include, for example, prescription drugs and related accessories that were prescribed to a patient by one or more physicians. Moreover, although a preferred and non-limiting embodiment concerns preparation and monitoring of a shipment (e.g., one or more packages) containing a single medication order from a pharmacy to a patient or caregiver (e.g., a final destination), those skilled in the art will appreciate that the systems and methods disclosed herein may also be used in connection with other types of shipments. For example, in some non-limiting embodiments, the system and method may be used to prepare and monitor bulk shipments of pharmaceuticals, such that the medication order includes a stock order for a distributor or multiple medication orders for multiple intended recipients. Moreover, non-limiting embodiments of the system and method described herein may also be used to prepare and monitor shipments of any type of perishable item, such as but not limited to food products.

In non-limiting embodiments, various medication order data associated with a medication order is recorded and monitored including, but not limited to, a patient name or identifier, an order number or identifier, one or more pharmaceutical components, one or more National Drug Code (NDC) numbers, a quantity, one or more lot numbers, a date the prescription was filled, a tracking number or identifier, one or more expiration dates, and/or other like medication order data pertaining to a patient, physician, and/or one or more prescribed medications. Medication order data may also include shipment data pertaining to the shipment of a medication order from a first location to a second location including, for example, a destination, a route, a timeframe, a mode of transportation, courier tracking information, geographic coordinates for the starting location, destination, and/or route, environmental conditions for the starting location, destination, and/or route, drug stability data, transportation duration, and/or other like information pertaining to the preparation, shipment, and/or delivery of a medication order.

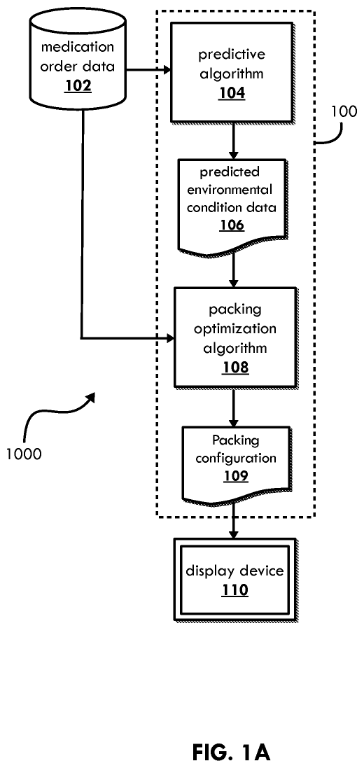

Referring now to FIGS. 1A and 1B, a system 1000 for dispensing and monitoring pharmacy shipments is shown according to a preferred and non-limiting embodiment. Medication order data 102 is stored in one or more data storage devices such as, for example, a hard disk or any other type of memory. The medication order data 102 may be arranged in any suitable data structure and may be local and/or remote to other components of the system 1000. In non-limiting embodiments, the medication order data 102 may be encrypted and/or de-identified in compliance with the Health Insurance Portability and Accountability Act (HIPAA) and/or other regulations. A computing device 100 is in communication with the medication order data 102. The computing device 100 includes one or more processors programmed or configured to execute one or more software applications. The computing device 100 may include, for example, one or more separate computers, mobile devices, server computers, and/or the like.

In non-limiting embodiments, and with reference to FIG. 1B, a predictive algorithm 104 may receive, as inputs, correlated temperature data 103. In such examples, a correlation is generated between the ambient outdoor temperature obtained from past shipment data 111 (as determined, for example, with a temperature sensor before, during, or after each past shipment) and historical environmental data 107 for specific transportation networks (e.g., shipping lanes). By generating a correlation between ambient conditions from previous shipment data 111 and historical environmental data 107, a more accurate ambient temperature can be predicted for a particular shipping lane that will be used to transport the package. In non-limiting embodiments, a correlation algorithm 105, executed by at least one processor, parses a plurality of past shipment data 111 (e.g., ISTA 7E shipment files or the like) and identifies the date and/or time stamp for the beginning of each previous shipment. This date and/or time data is then used by the correlation algorithm 105 to match the past shipment data 111 to historical environmental data 107 and, using both sets of data, generate correlated temperature data 103. Historical environmental data 107 may be obtained and compiled from one or more government agencies and/or third-party weather services.

In non-limiting embodiments, and with continued reference to FIG. 1B, the correlation algorithm 105 may generate the correlated temperature data 103 using a step-wise multiple linear regression analysis or other statistical analysis techniques. In some examples, a brute force method may be implemented in which every available independent variable is used for the linear regression analysis. In this manner, linear regression analysis allows for the identification of independent variables that are correlated to other variables. If a high correlation between independent variables is determined, those variables are removed from the analysis. One or more of the least statistically significant variables may then be removed and the linear regression analysis repeated until only statistically significant variables remain in the model. In non-limiting examples, the least statistically significant variable may be determined by analyzing the probability that the calculated coefficient for xᵢ is a nonzero value. In some examples, each cycle removes a single least statistically significant variable, although it will be appreciated that several variables may also be removed at once.

In some non-limiting embodiments, instead of or in addition to the brute force method, the correlated temperature data 103 may be generated with a scientific method step-wise multiple linear regression analysis by first removing any independent variables from the data set that are deemed to be irrelevant or less impactful for the model, such as but not limited to wind direction, mean sea level pressure, and precipitation amount. It will be appreciated that other independent variables may be removed and that, in some examples, the removed independent variables may be identified using analytic techniques to determine an amount of impact that each variable may have. Once these variables are removed from the data set, the step-wise linear regression analysis proceeds in the same manner as the brute force method described above. In some non-limiting embodiments, correlations may be determined by the correlation algorithm 105 with a curvilinear regression analysis.

Still referring to FIG. 1B, regardless of the manner in which the statistical analysis is performed by the correlation algorithm 105, the outputs of the statistical analysis (e.g., such as the outputs of each linear model in embodiments in which a linear regression analysis is performed) is thereafter processed by the correlation algorithm 105 to generate the correlated temperature data 103. For example, the number of variables present in each model, the specific variables present in each model, and the Pearson correlation coefficient of each model may be determined by a processor executing the correlation algorithm 105. The Pearson correlation coefficient (R² value) represents the linear correlation between variables (e.g., how well the independent variables in the model explain the variation between variables).

Based on the multiple linear regression analysis or other statistical analysis technique, correlations may be determined between ambient transportation temperatures (identified from past shipment data 111) and historical environmental data 107 to generate the correlated temperature data 103. Through the examination of individual shipment models, specific lane models, global models, and/or an average value model, predicted ambient transportation temperatures can be determined from the correlated temperature data 103 based on the use of specific lane models and the average value model. In FIG. 1B, the correlation algorithm 105 is depicted as being external to the computing device 100. However, it will be appreciated that, in some non-limiting embodiments, the correlation algorithm 105 may be executed by one or more processors of the computing device 100 or one or more different computing devices that are local or remote to the computing device 100.

The following table depicts variables corresponding to environmental condition parameters that may be used in the statistical models described herein:

Variables Used in Statistical Model

| Origin Variables | Destination Variables |
|---|---|
| O-Surface Temperature Celsius | D-Wind Speed Kph |
| O-Cloud Coverage Percent | D-Surface Air Pressure Kilopascals |
| O-Precipitation Previous Hour | D-Apparent Temperature Celsius |
| Centimeters | |
| O-Surface Air Pressure Kilopascals | D-Precipitation Previous Hour Centimeters |
| O-Downward Solar Radiation Wsqm | D-Downward Solar Radiation Wsqm |
| O-Direct Normal Irradiance Wsqm | D-Msl Pressure Kilopascals |
| O-Diffuse Horizontal Radiation Wsqm | D-Surface Wet Bulb Temperature Celsius |
| O-Msl Pressure Kilopascals | D-Surface Wind Gusts Kph |
| O-Wind Direction Degrees | D-Wind Direction Degrees |
| O-Surface Wind Gusts Kph | D-Direct Normal Irradiance Wsqm |
| O-Surface Dewpoint Temperature Celsius | D-Surface Dewpoint Temperature Celsius |
| O-Surface Wet Bulb Temperature Celsius | D-Relative Humidity Percent |
| O-Relative Humidity Percent | D-Diffuse Horizontal Radiation Wsqm |
| O-Apparent Temperature Celsius | D-Cloud Coverage Percent |
| O-Wind Speed Kph | D-Surface Temperature Celsius |
| O-Snowfall Centimeters | D-Snowfall Centimeters |

In some non-limiting embodiments, and with continued reference to FIG. 1B, the correlation algorithm 105 may be executed with respect to each shipment. The correlation algorithm 105 may, for example, receive forecasted environmental data 113 (as obtained from a third-party weather service) and actual environmental data 112 as inputs. It will be appreciated that the correlation algorithm 105 may be executed for each shipment before, during, and/or after the shipment. Correlations that are determined may be stored as correlated temperature data 103 for improving future performance of the predictive algorithm 104. In some examples, the correlation algorithm 105 that receives forecasted environmental data 113 and actual environmental data 112 may be part of the predictive algorithm 104. It will be appreciated that various arrangements are possible and that the correlation algorithm 105 and predictive algorithm 104 may be implemented by one or more software applications.

In a non-limiting embodiment, and with continued reference to FIGS. 1A and 1B, the computing device 100 implements a predictive algorithm 104 for processing the medication order data 102 and, in some examples, the correlated temperature data 103 (shown in FIG. 1B). The predictive algorithm 104 may be implemented through a software application executed by one or more processors of the computing device 100 (e.g., as a local client application) or on a remote server (e.g., as a software-as-a-service application). The predictive algorithm 104 may be programmed or configured to determine predicted environmental condition data 106 for a medication order that is to be shipped from a first location to a second location. The predicted environmental condition data 106 may include predicted values for one or more environmental condition parameters such as, for example, temperature, humidity, pressure, and/or the like. In a preferred and non-limiting embodiment, the predicted environmental condition data 106 relates to predicted conditions inside of a package containing a particular medication order during shipment. For example, the predicted environmental condition data 106 may relate to an interior temperature of a transportation unit (e.g., a vehicle or cargo container), package, or pharmaceutical. However, it will be appreciated the predicted environmental condition data 106 may also relate to predicted ambient conditions that the exterior of the package will be exposed to during shipment.

With continued reference to FIGS. 1A and 1B, and in a non-limiting embodiment, the predictive algorithm 104 may process, as inputs, one or more thermodynamic properties of the medication packaging, geographic coordinates (e.g., longitude and latitude) for the first location, second location, and/or route between both locations, mode of transportation, transportation route, shipping lanes, actual environmental conditions, historic environmental conditions, and/or other like information that may impact the environmental conditions that the medication order may be exposed to during transportation from the first location to the second location. Some or all of this data may be obtained from medication order data 102 and/or correlated temperature data 103, although it will be appreciated that the input data may be obtained from various systems and sources.

In a preferred and non-limiting embodiment, the first location is a source pharmacy and the second location is a patient's residence or location, but it will be appreciated that the system 1000 and method disclosed herein may also be used to package and monitor medication orders transported between any locations. Further, the predictive algorithm 104 may include thermodynamic formulas for estimating environmental conditions within a package based on the inputs to the algorithm. In a preferred and non-limiting embodiment, the predictive algorithm 104 uses one or more coefficients associated with the packaging materials as inputs to determine the predicted environmental condition data 106.

In a preferred and non-limiting embodiment, the predictive algorithm 104 operates in multiple stages. A first stage determines a predicted effect that ambient environmental conditions, such as temperature, will have on an interior of a transportation unit (e.g., a vehicle or cargo container). This may use, as inputs, an ambient temperature as a function of time, latitude, and longitude, a surface area of the transportation unit, a volume enclosed by the transportation unit, a wall thickness of the transportation unit, and a coefficient of heat of the material used for the wall of the transportation unit. This stage of the predictive algorithm 104 may output a predicted temperature or other environmental condition for inside the transportation unit as a function of time (e.g., T(t)ᵤₙᵢₜ).

In a preferred and non-limiting embodiment, a second stage of the predictive algorithm 104 determines a predicted effect that the environmental conditions of the transportation unit will have on a parcel. A parcel may include, for example, a box or other type of shipping container. In this second stage, the predicted temperature of the inside of the parcel may be determined in two ways and averaged. For example, a first estimated temperature inside the parcel as a function of time (e.g., T(t)_{parcelA}) may be determined based on inputs including an average temperature of a cooling pack (e.g., ice unit or gel pack) as a function of time (e.g., T(t)_{ice}), a surface area of the cooling pack (A_{ice}), a volume of the cooling pack (V_{ice}), a volume enclosed by the parcel (V_{parcel}), a thickness of the cooling pack (t_{ice}), and a coefficient of heat of the cooling pack (C_{h,ice}). A second estimated temperature inside the box as a function of time (e.g., T(t)_{parcelB}) may be determined based on inputs including the predicted temperature inside the transportation unit as a function of time (e.g., T(t)ᵤₙᵢₜ) determined in the first stage, a surface area of the parcel (A_{parcel}), a volume enclosed by the parcel (V_{parcel}), a thickness of the walls of the parcel (t_{parcel}), and a coefficient of heat of the parcel (C_{h,parcel}). Both predicted temperatures (T(t)_{parcel}A and T(t)_{parcelB}) may then be averaged to arrive at T(t)_{parcel}.

In a preferred and non-limiting embodiment, a third stage of the predictive algorithm 104 determines a predicted effect that the environmental conditions of the parcel will have on the surface of a pharmaceutical. This stage is applicable when the pharmaceutical is wrapped in a final layer of insulating material. The estimated temperature inside of the insulating material over time (e.g., T(t)ᵢₙₛᵤₗₐₜᵢₒₙ) is determined based on inputs including the estimated temperature inside the box as a function of time (e.g., T(t)_{parcel}), as determined in the previous stage, the surface area of the insulating material (Aᵢₙₛᵤₗₐₜᵢₒₙ), the volume enclosed by the insulating material (Vᵢₙₛᵤₗₐₜᵢₒₙ), the thickness of the insulating material (tᵢₙₛᵤₗₐₜᵢₒₙ), and a coefficient of heat of the insulating material (C_{h,insulation}). It will be appreciated that the predicted environmental conditions inside of the package may be determined in any number of other ways.

In non-limiting embodiments, the predictive algorithm 104 utilizes modeling techniques to represent conductive heat transfer within thermal packaging materials. A first order model may be used to estimate a first order process, such as conductive heat transfer. Conductive heat transfer may be represented by Fourier's Law: dQ/dt=kAΔT. The general style of a first order model is: τₚdy(t)dt=−y(t)+Kₚx(t−θp), where y(t) and x(t) are variables and the parameters are: Kₚ=process gain; τₚ=process time constant; and θp=process dead time. To simplify the solution of a first order model, a Laplace transform may be performed. The resultant equation is referred to as a transfer function. The transfer function for a first order plus dead time model is: Y(s)/X(s)=Kₚe^{-θps}/(τₚs+1). Using these calculations, the three above-described heat transfer processes may be simulated. Additional considerations may be appropriate for calculating the temperature of a cooling pack when frozen and after undergoing a phase change.

Using modeling tools, such as but not limited to MATLAB and Simulink, three dynamic function blocks may be generated for each of three heat transfer processes that may be analyzed by the predictive algorithm 104: (1) ambient environment to product (e.g., inside of a parcel, such as a medication order), (2) ambient environment to cooling pack (e.g., ice), and (3) cooling pack to product. Each dynamic block may be represented by a first order transfer function and each dynamic block may have an associated dead time block (e.g., a secondary block used to delay information communicated to other blocks) inputted into the transfer function. As described herein, the predictive algorithm 104 may receive, as inputs, correlated temperature data. This correlated temperature data may represent a correlation between actual environmental data and forecasted environmental data.

In some non-limiting examples, a fourth dynamic block may be generated and incorporated into the predictive algorithm 104 to model the phase change that occurs in the cooling pack throughout a simulated shipment. The fourth dynamic block may be an integral block and may be used to determine a total heat transferred. The total heat transferred may be compared to an amount of latent heat present in the cooling pack and the compared value may be inputted into a logical function block (e.g., an if/else statement) to determine how to calculate the current temperature of the cooling pack.

In non-limiting embodiments, an ambient temperature profile is inputted into the model. In particular, the ambient temperature profile may be inputted into the dynamic block representing the heat transfer process of ambient environment to product and the dynamic block representing the heat transfer process of ambient environment to cooling pack. The outputs from these dynamic blocks are input into the integral block to determine a total heat transferred. Once a new temperature of the cooling pack is determined, the new temperature is input into the final transfer function block representing the third heat transfer process from cooling pack to product. The model may then output a sum of the product temperature results.

In some non-limiting embodiments, the transfer function parameters may be estimated once the model is developed. As an example, the following table shows estimated parameters that may be used in each transfer function in non-limiting embodiments involving two different types of product packaging:

| | | 1.5″ Molded | | | 1.0″ Cut Sheet | | |
|---|---|---|---|---|---|---|---|
| | | EPS Packaging | | | EPS Packaging | | |
| | | Kₚ | Θp | τₚ | Kₚ | θp | τₚ |
| Heat | Ambient | 0.1 | 1 | 5 | 0.1 | 1 | 20 |
| Transfer | Environment | | | | | | |
| Process | to Product | | | | | | |
| | Ambient | 1 | 1 | 75 | 1 | 1 | 20 |
| | Environment | | | | | | |
| | to Cooling | | | | | | |
| | Pack | | | | | | |
| | Cooling Pack | 3.6 | 1 | 40 | 2.5 | 1 | 70 |
| | to Product | | | | | | |

It will be appreciated that the estimated transfer function parameters may vary depending on the type of packaging used, the type of cooling pack used, and/or other variables.

With continued reference to FIGS. 1A and 1B, the computing device 100 may also implement a packing optimization algorithm 108 through a software application executed by one or more processors of the computing device 100 or on a remote server. The packing optimization algorithm 108 may process, as inputs, medication order data 102 and/or the predicted environmental condition data 106. In a preferred and non-limiting embodiment, the packing optimization algorithm 108 processes drug stability data, predicted environmental condition data 106, actual environmental data, a quantity and/or size of the medication order, an expected transportation duration, types of available cooling packs or other available packaging materials, and the mode of transportation. Based on these inputs, the packing optimization algorithm 108 then generates a packing configuration 109 reflecting a recommended and/or optimal configuration of the package(s) in which the medication order is to be shipped. The packing configuration 109 may reflect, for example, a package size, a type of box or other packaging materials, a cooling pack (e.g., gel or ice pack) configuration for maintaining the medication order at a desired temperature, an arrangement of items that belong in the package, and/or other like information concerning the packaging of the medication order.

Still referring to FIGS. 1A and 1B, the packing configuration 109 may be dynamically generated based on the medication order data 102, predicted environmental condition data 106, and/or other inputs. In other non-limiting embodiments, the packing configuration 109 may be selected from a plurality of predefined configurations, such as packing configuration templates, based on a closest fit. The packing configuration 109 may be a structured data format, textual format, and/or graphical format. A visual representation of the packing configuration 109 is generated and displayed on a display device 110 at a packaging station to instruct an individual how to configure the packaging of the medication order shipment. As an example, a visual representation of a packing configuration 109 may be displayed as one or more diagrams, animations, videos, textual narratives, step-by-step instructions, and/or the like. The packing configuration 109 may also be stored in a data storage device such that it can be later referenced, used to influence and/or modify the packing optimization algorithm 108, or to verify compliance with specified regulations or parameters. Moreover, an image capture device may be used to capture one or more images of the packaged and/or unpackaged medication order for record keeping and/or compliance.

The packing optimization algorithm 108 may determine the type of exterior packaging materials to use to ship the medication order (e.g., a box) and the type and/or configuration of the packaging and insulating materials used within the exterior packaging materials. In a preferred and non-limiting embodiment, the packing optimization algorithm 108 may operate in multiple stages. The first stage of the packing optimization algorithm 108 may process, as inputs, a desired temperature inside the package as a function of time (T(t)_{parcel,desired}) and the average temperature of the transportation unit as a function of time (T(t)ᵤₙᵢₜ), as determined by the predictive algorithm 104. Based on these inputs, the first stage of the packing optimization algorithm 108 may output a volume enclosed by the parcel (V_{parcel}), a surface area of the parcel (A_{parcel}), a thickness of the parcel wall (t_{parcel}), and a coefficient of heat of the parcel (exterior packaging material) (C_{h,parcel}). From these outputs, a type of parcel may be selected that is a closest fit to the optimal parameters.

The second stage of the packing optimization algorithm 108 may process, as inputs, a desired temperature inside the package as a function of time (T(t)_{parcel,desired}) and a desired temperature inside the insulating material as a function of time (T(t)_{insulation,desired}). Based on these inputs, the second stage of the packing optimization algorithm 108 may output a volume of the cooling pack (V_{ice}), a surface area of the cooling pack (A_{ice}), a thickness of the cooling pack (t_{ice}), a coefficient of heat of the cooling pack C_{h,ice}), a volume enclosed by the insulating material (Vᵢₙₛᵤₗₐₜᵢₒₙ), a surface area of the insulating material (Aᵢₙₛᵤₗₐₜᵢₒₙ), a thickness of the insulating materials (tᵢₙₛᵤₗₐₜᵢₒₙ), and a coefficient of heat of the insulating material (C_{h,insulation}). From these outputs, insulating materials may be selected that are a closest fit to the optimal parameters.

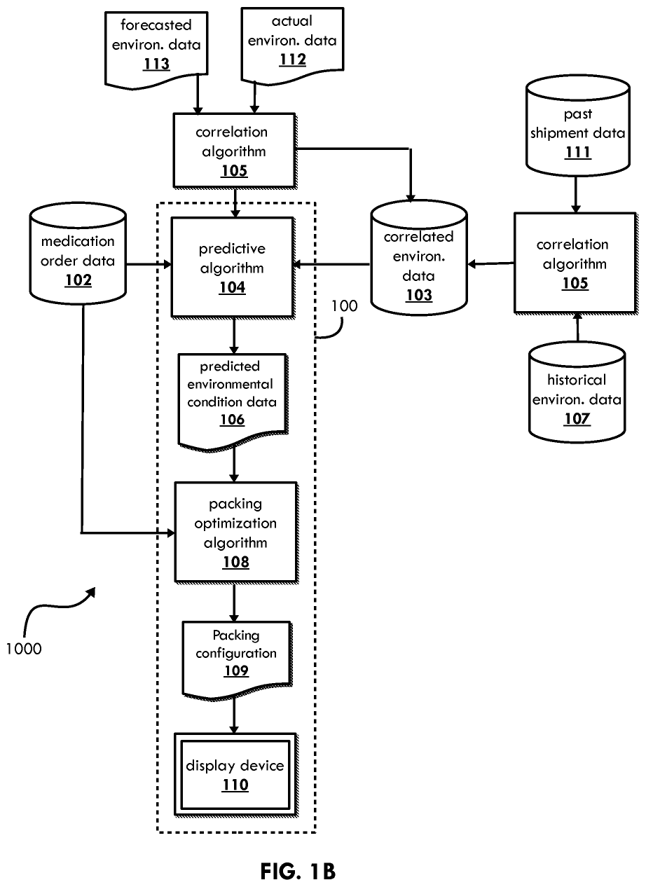

Referring now to FIG. 2, a system 1000 for monitoring pharmacy shipments is shown according to a preferred and non-limiting embodiment. A package 101 containing a medication order is prepared and shipped using a desired mode of transportation. In some non-limiting examples, the package 101 may include one or more sensors 118 and data sources 117. A sensor 118 may include one or more temperature, humidity, or pressure sensors, as examples. A data source 117 may include one or more barcodes (e.g., one- or two-dimensional barcodes), RFID transponders, solid-state memories, machine-readable characters, and/or the like. In a preferred and non-limiting embodiment, the data source 117 comprises a unique identifier that is linked in a database to medication order data for a particular patient and/or shipment. In this manner, a single data source 117 may be used to determine or identify data that may be separately encoded in a courier data source (such as a shipment or tracking barcode) and/or in a pharmaceutical manufacturer data source (such as barcodes on the medications or other items in the package 101). In addition to linking to data stored in a database, the data source 117 itself may also include medication order data such as, for example, a patient name or identifier, one or more prescription numbers or identifiers, one or more order numbers or identifiers, one or more drug component identifiers, a quantity of medication, one or more lot numbers, one or more National Drug Code (NDC) numbers, one or more expiration dates, a courier tracking identifier, and/or other medication order data. In non-limiting embodiments, the medication order data on the data source 117 may be encrypted and/or de-identified. The data source 117 may also be used to link the package 101 to the patient, physician, and/or physician's office.

In a preferred and non-limiting embodiment, the environmental condition data 112 collected by the sensor 118 is used to train, influence, and/or test the predictive algorithm 104 and/or the correlation algorithm 105. The sensor 118 may detect and/or measure environmental conditions during transportation of the package 101 including, for example, temperature, humidity, pressure, and/or the like. The sensor 118 may either be located in or on the package 101, in a vehicle transporting the package 101, in a warehouse storing the package 101, or in a mobile device used by a delivery agent. In a preferred and non-limiting embodiment, the sensor 118 is located inside the package. The environmental condition data 112 collected by the sensor may be stored locally on memory or may be communicated to a remote server or device for storage. In a preferred and non-limiting embodiment, the environmental condition data 112 is stored on memory local to the sensor and is retrieved at a later time, such as when the recipient or courier returns the sensor. In other non-limiting embodiments, the sensor 118 includes or is in communication with a wireless communication device that periodically communicates the environmental condition data 112 to a remote server or device. Further, the sensor 118 may include a wireless or wired network interface to transmit the environmental condition data 112 over an Internet connection. It will be appreciated that various other arrangements are possible.

Once the predictive algorithm 104 is trained, influenced, and/or developed, use of the sensor 118 may be phased out and no longer used. In some non-limiting embodiments, the sensor 118 may not be used for any shipments and the predictive algorithm 104 may operate independently of actual sensor data. In other non-limiting embodiments, the sensor 118 may be included in some or all shipments, even after the predictive algorithm 104 is trained, influenced, and/or developed, to maintain a log of actual environmental conditions. It will be appreciated that various other arrangements are possible.

In non-limiting embodiments, the environmental condition data 112 collected by the sensor 118 may be correlated to forecasted environmental data 113 and the resulting correlated data may be stored as correlated environmental data 103 in a database. In this manner, the correlated environmental data 103 improves over time and, as a result, improves the accuracy of predictions generated by the predictive algorithm 104.

With continued reference to FIG. 2, a compliance algorithm 116 may be used to process the environmental condition data 112 and compliance condition data 114 to determine whether transportation of the package 101, including the conditions experienced during transportation, complied with one or more predetermined regulations or rules. The compliance condition data 114 may be predefined values or ranges for one or more environmental condition parameters, and may be hosted by the system 1000 or obtained from a government agency or third party. The compliance algorithm 116 may determine compliance with one or more rules or regulations after the package 101 has been delivered to a patient and/or during transportation of the package 101. In a non-limiting embodiment, the compliance algorithm 116 compares values of one or more environmental condition parameters from the collected environmental condition data 112 to the compliance condition data 114 to determine whether the actual environmental condition data 112 falls within a predefined or specified range of the compliance condition data 114.

In some non-limiting embodiments, rather than or in addition to comparing environmental condition data 112 to the compliance condition data 114, the compliance algorithm 116 may compare shipping data collected during transportation of the medication order such as, for example a transportation time, a route, a delay, an exposure time, and/or the like, to compliance condition data 114. In some examples, the compliance algorithm 116 may compare the compliance condition data 114 and the environmental condition data 112 and/or shipping data at discrete instances during shipment (e.g., comparing the data when an exposure to the environment or a shipment delay occurs) or in the aggregate over the entire shipment time. For example, if multiple environmental exposures or delays occur but each individual exposure or delay does not itself exceed one or more values of the compliance condition data 114, multiple individual exposures or delays, in the aggregate, may still result in noncompliance. Thus, one or more parameters of the environmental condition data 112 and/or shipping data may be cumulated during shipping and the cumulative value compared to the compliance condition data 114. As an example, if a first event (e.g., exposure, delay, or the like) results in a nominal degradation of a medication that does not trigger an alert or result in noncompliance, the degradation amount may be added to through additional events experienced during shipment and the aggregate degradation amount may be compared to compliance condition data 114. Those skilled in the art will appreciate that various other arrangements are possible.

In a preferred and non-limiting embodiment, the compliance algorithm 116 may receive, as inputs, the predicted internal average temperature of the insulating material as a function of time (T(t)ᵢₙₛᵤₗₐₜᵢₒₙ) and a threshold temperature of the pharmaceutical as a function of time (T(t)_{threshold}). Further, in non-limiting embodiments, an actual internal average temperature, as obtained from one or more sensors 118, may be used instead of a predicted internal average temperature. The threshold temperature may be obtained from the compliance condition data 114. The compliance algorithm 116 may have a binary output (e.g., true or false) that can be used to indicate that the shipment complied or trigger an alert to indicate that the shipment did not comply. It will be appreciated that various other arrangements and calculations are possible.

The collected environmental condition data 112 may include values for one or more environmental condition parameters such as, for example, temperature, humidity, pressure, and/or other like parameters, and each value may be associated with a shipment, route, location, date, and/or the like. For example, an environmental condition parameter may have a value representing an environmental condition at one particular location (e.g., a starting location, a point along a route, or a destination location) or an average of values for different locations and/or times. The compliance condition data 114 may include predefined values or ranges of values for environmental condition parameters provided by a manufacturer, a government agency, a pharmacy, and/or a third party, as examples. The compliance condition data 114 may also include ranges or values to be used as thresholds for generating and transmitting alerts. Different ranges or values may be used for determining what type of alerts to generate (e.g., compliance, non-compliance, warnings, suggestions for improvement, and/or the like) and who should receive the alerts (e.g., a patient, a physician, a caregiver, a pharmacy, and/or the like). In non-limiting embodiments, the values or ranges of the compliance condition data 114 are based upon the stability of a medication and the environmental conditions that could affect such stability.

Still referring to FIG. 2, if the compliance algorithm 116 determines that the collected environmental condition data 112 is not compliant with the compliance condition data 114, it may generate an alert. The alert may be transmitted to a remote device 119, such as a mobile phone (via text message, phone call, media message, push notification, or the like), a laptop, a server, or any other remote device 119 capable of receiving an alert. An alert may also be generated and transmitted in response to determining that the collected environmental condition data 112 is compliant with the condition data 114. The remote device 119 may also log compliance data in a database. In some non-limiting examples, the remote device 119 may include a server computer programmed or configured to generate a compliance report concerning one or more shipments. A compliance report may be arranged by package, medication order, shipping carrier, route, patient, pharmacy, and/or the like.

In non-limiting embodiments, the actual environmental condition data 112 may be used to improve and influence the predictive algorithm 104, packing optimization algorithm 108, and/or compliance algorithm 116. For example, machine-learning techniques may be implemented to make the predictive algorithm 104, packing optimization algorithm 108, and/or compliance algorithm 116 iteratively self-learning by adjusting and/or refining the algorithms 104, 108, 116 based on actual data and prior outputs of the algorithms 104, 108, 116. In this manner, as shipping data, medication order data, and compliance data is accumulated and aggregated, the algorithms 104, 116 can be influenced by this data by being adjusted to improve efficiencies. For example, if a particular packaging configuration is found to be unsuccessful in achieving compliance, an alternative packaging configuration may be identified based on a previously compliant shipment for a medication order with a common or similar parameter, such as route, travel time, environmental conditions, and/or the like. Over time, the algorithms 104, 116 may identify different parameters that impact the environmental conditions differently.

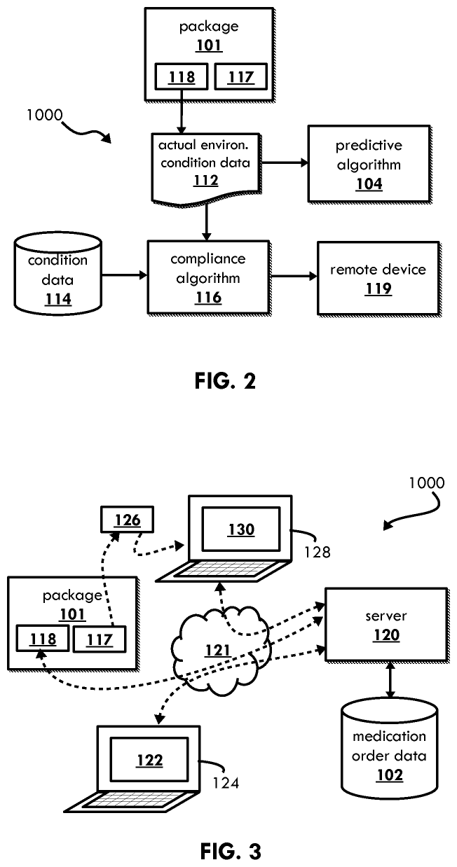

FIG. 3 shows a system 1000 for packaging and monitoring pharmacy shipments according to a preferred and non-limiting embodiment. A client device 124 is in communication with a server computer 120 via a network environment 121. The sensor 118 associated with the package 101 may also be in communication with the remote server 120 via the network environment 121 or a different network environment. The network environment 121 may be, for example, a private or public network, a local area network, a wide area network, the Internet, a cellular network, and/or any other like network environment. The client device 124 may be, for example, a computer terminal, laptop, mobile device, or other like computing device. The client device 124 presents a graphical user interface (GUI) 122 to facilitate a user to input and/or edit medication order data 102 for one or more patients. For example, the GUI 122 may be a secure, web-based interface accessed on the client device 124 that includes various selectable options to input a medication order for a patient. Selectable options may include, for example, one or more buttons, checkboxes, drop-down menus, input fields, decision support menus, and/or the like. The client device 124 and GUI 122 may be used by a physician, healthcare administrator, or insurance company, as examples, to access the medication order data 102. In a preferred and non-limiting embodiment, the GUI 122 is used by a pharmacist to verify the medication order prior to shipment.

With continued reference to FIG. 3, various rules and/or permissions may be implemented to control access to and/or modification of the medication order data 102. A second client device 128 may receive medication order data from one or more data sources 117 on or associated with the package 101. For example, a data receiving device 126, such as an RFID reader, barcode scanner, camera, or the like, may receive a medication order identifier from the data source 117 at the pharmacy, during transportation, and/or at delivery. The client device 128 may then look up additional medication order data 102 using the unique identifier. As an example, the client device 128 may receive medication order data 102 by communicating a request to the server 120 that includes a unique medication order identifier. In some examples, the unique identifier may be used to track the package 101 such that the location of the client device 128 is associated with the package 101 at a particular time (e.g., the package has arrived at or departed a particular location at a particular time). The data source 117 may allow for the system 1000 to track and identify the package 101 as it passes through multiple facilities and locations, including packaging, transportation, storage, and the like. The second client device 128 may display a GUI 130 that allows for the package 101 to be verified and for at least a portion of the medication order data to be confirmed or edited. In some examples, the data receiving device 126 and second client device 128 may be a single device, such as a smartphone or mobile computer. In non-limiting embodiments, the second client device 128 may be used by a shipping carrier to scan and track the package 101 throughout the shipment process. The second client computer 128 may also be in communication with the sensor 118 to obtain environmental condition data before, during, or after transportation.

Still referring to FIG. 3, the second client device 128 may be in communication with the server 120 through the network environment 121 or a different network environment. The second client device 128 may communicate shipment data, medication order data obtained from the data source 117, and/or environmental condition data obtained from the sensor 118 before, during, or after transportation of the package 101. Shipment data may include a subset of medication order data such as, for example, a location of the package 101, an identification of a courier, an estimated time of arrival, and/or other like information concerning the transportation of the package 101. The shipping data may be input by a user into the second client device 128 or may be obtained in other ways. Although FIG. 3 illustrates two client devices 124, 128, those skilled in the art will appreciate that any number of client devices may be used in the system 1000 and that different users and/or devices may be associated with different permissions defining what actions they can take and/or what information can be accessed.

The server 120 may execute one or more software programs for enabling various functions of the system 1000. For example, an analytics software program may utilize an Application Programming Interface (API) to access the medication order data 102. The software program may provide for real-time and/or historical analytics, configuration of alerts and/or trigger events, report generation, and/or the like through the use of one or more GUIs, such as a visual dashboard. The GUIs may include various selectable options for a user to specify parameters. For example, a user may be able to assign rules to trigger events by designating a value or range such that, when that particular parameter meets, exceeds, or falls below the specified value or range, depending on the rule selected by the user, an alert is generated. Users may also configure the system 1000 to generate alerts in response to the package being present at a particular location, a warning or recall from a manufacturer for one or more medications in the shipment, or any other specified condition. Users may also configure the system 1000 to specify individuals or entities that will receive the alerts.

In non-limiting embodiments, the server 120 may execute one or more software programs and/or functions for providing decision support and to facilitate documentation of decisions and other information relating to the medication order. For example, a decision support menu may guide a user through one or more GUIs for utilizing one or more functions of the system 1000. Users may also document why and/or how they make certain decisions, such as approving or disapproving an order for shipment or reshipment, modifying shipment data, setting up an alert, and/or the like. This information may be provided through one or more selectable options, such as drop-down menus or text input boxes, and stored as medication order data 102 or in any other database. As an example, a decision support tool may display a visual representation of medication order data and/or shipment data as text, graphs, charts, and/or the like, to facilitate a user's decision of whether to approve or disapprove a shipment. It will be appreciated that other arrangements are possible.

In non-limiting embodiments, the system may be used to alert patients, caregivers, or other individuals to prevent the consumption of recalled, illegitimate, expired, and/or adulterated medications. For example, if a medication is determined to be undesirable for any reason (e.g., expired, recalled, adulterated, etc.), the shipment can be stopped in transit or the patient notified prior to consumption. By including the expiration date and lot number in the medication order data, the system can identify all medication orders that include an undesirable medication. Other parameters may also be used, such as an NDC, medication identifier, pharmacy identifier, or the like. Moreover, alerts may also be generated in response to determining that the environmental conditions during transport did not comply with compliance data. Alerts may also be generated after delivery of the medication order, during the course of therapy for example. In non-limiting embodiments, reshipments may be automatically triggered in response to determining that a medication is recalled, illegitimate, expired, and/or adulterated. Alerts may also trigger scheduled appointments to follow-up with a patient regarding replacement medications and/or packages.

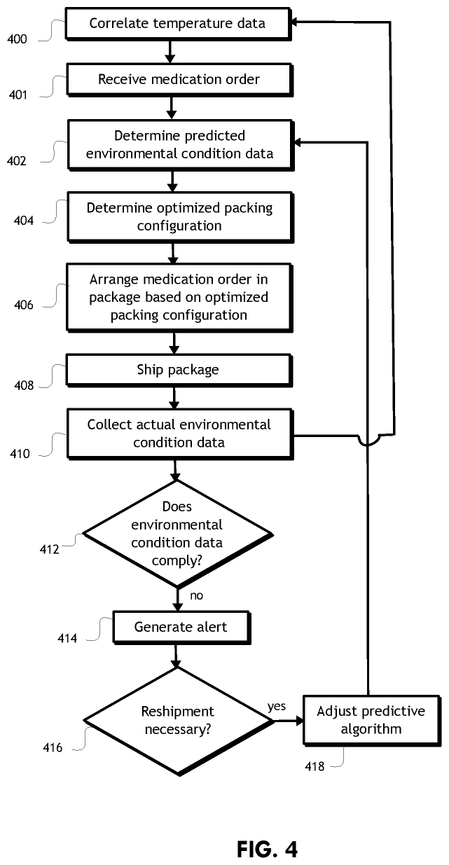

Referring now to FIG. 4, a flow diagram is shown for a method of packaging and monitoring pharmacy shipments according to a preferred and non-limiting embodiment. At a first step 400, environmental condition data is correlated. For example, a correlated temperature database may be created by processing historical environmental data and past shipment data with a correlation algorithm. At step 401, a medication order is received. At a next step 402, predicted environmental condition data is determined. The predicted environmental condition data includes values for environmental condition parameters that are expected during transportation of the medication order from a first location, such as a pharmacy, to a destination location, such as a patient or caregiver. At step 404, an optimized packing configuration is determined. The optimized packing configuration may be based on the predicted environmental condition data and other parameters. At a next step 406, the medication order is arranged in a package according to the optimized packing configuration. Step 406 may be performed by an individual that is being instructed by a display device or, in other examples, may be performed automatically by a robotic packing device.

With continued reference to FIG. 4, the package is shipped to the destination location at step 408. During shipment, in some non-limiting embodiments, actual environmental condition data may be collected with one or more sensors at step 410. At step 410, the actual environmental condition data may be fed back into the correlation algorithm at step 400 to correlate the data with forecasted environmental data, therefore improving the accuracy of future predictions. After or during shipment, at step 412, it is determined whether the environmental conditions experienced during shipment comply with predefined compliance parameters. If the conditions do comply, the method may end or, in other examples, proceed to an additional step involving adjusting the predictive algorithm used to determine the predicted environmental condition data based on the actual environmental condition data. If the conditions do not comply at step 412, the method may proceed to step 414 in which an alert is generated and/or transmitted. At step 416, it is determined whether reshipment is necessary. If reshipment is necessary, the method may adjust the predictive algorithm and/or correlation algorithm through feedback data obtained through the process (e.g., actual environmental condition data, forecasted environmental condition data, predicted environmental condition data, compliance results, and/or the like) at step 418 and then proceed to step 402 to determine predictive environmental condition data and restart the packaging process for the reshipment. The predictive algorithm and/or correlation algorithm may also be adjusted after any of steps 412, 414, 416, or at any other time that relevant data is available. It will be appreciated that, during or between any of steps 408, 410, 412, 414, and 416, the recipient (e.g., patient) may receive the package and the recipient and/or the courier may scan the package (e.g., scan a label on the package having machine-readable indicia) to indicate that the delivery is complete. Upon scanning the package, a notification may be sent to a remote server with or without any data obtained from sensors.

Non-limiting embodiments of the present invention may provide a source of clinically relevant, real-time quality information that can be used to improve operational efficiencies, patient safety, and therapy outcomes. These improvements may include, for example, efficiencies at the packaging station, reduction of full time equivalents (FTEs) (e.g., hours worked by a full-time employee), decrease of packing supply waste and usage, decrease in shipping costs, elimination of unnecessary and non-reimbursed reshipments, improvement of patient satisfaction, increased compliance with manufacturer stability recommendation, increased compliance with regulatory and certification recommendations, maximization in insurance reimbursement through enhanced services and documentation of a standardized chain of custody process, enhanced patient safety and therapy outcomes by eliminating or reducing temperature and storage as a variable, and other benefits.

In non-limiting embodiments, return shipments from the patient back to the pharmacy or some other destination may also be managed and tracked. For example, if a medication in a medication order is determined to be expired, adulterated, or recalled, the return shipment from the patient or caregiver back to the pharmacy or supplier may be tracked to ensure that the medications are accounted for and properly disposed of. One or more data sources may be provided to the patient to include on the outside of the return package. For example, a barcode may be provided that includes medication order data relating to the return shipment.

Non-limiting embodiments of the system may be implemented on a mobile application platform accessible to patients, pharmacies, physicians, shipping carriers, and/or insurance providers for sharing captured data. Different types of users may be presented with different GUIs and selectable options. For example, physicians may be provided with tools to access prescriptions for patients. Patients may be provided with tools to view their prescriptions and orders, costs, health information, and the like. Pharmacists may be provided with tools to contact or communicate with the physician and/or patient, interact with insurance companies, access prescriptions, verify medication orders, and/or the like. Users may access different components of the system through a web-based interface, a mobile application, a client-side software application, a server-side software application, and/or the like. It will be appreciated that various existing platforms may be utilized to integrate and connect different devices, software applications, and data sources including, for example, FHIRE, SMART Platform, HL7, XML, APIs, Webservices and Direct. Secure communication channels may be used for alerts, secure messaging, and the transmission of protected clinical or healthcare content.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A computer-implemented method for predictively monitoring a shipment of at least one medication order from a first location to a destination location, comprising:
receiving a medication order comprising medication order data identifying at least one medication for a patient;
determining, with at least one processor, predicted environmental conditions for the shipment of the at least one medication from the first location to the destination location based at least partially on the medication order data;
determining, with at least one processor, an optimized packing configuration of at least one of a cooling pack and insulating material for the at least one medication order based at least partially on the predicted environmental conditions; and
generating a visual representation of the optimized packing configuration on a display device.

2. The computer-implemented method of claim 1, further comprising:
collecting environmental condition data during shipment of the at least one medication from the first location to the destination location; and
determining whether the shipment of the at least one medication complies with predetermined transportation conditions by comparing the collected environmental condition data to the predetermined transportation conditions.

3. The computer-implemented method of claim 2, wherein the predicted environmental conditions are determined based at least partially on at least one of the following: a thermodynamic property of a packaging material, a geographical coordinate, an environmental condition, a shipping route, a mode of transportation, or any combination thereof.

4. The computer-implemented method of claim 3, wherein the predicted environmental conditions are determined based at least partially on at least one thermodynamic property of a packaging material.

5. The computer-implemented method of claim 2, wherein the predicted environmental conditions are determined based at least partially on a predictive algorithm, and wherein the predictive algorithm is modified based on the collected environmental condition data.

6. The computer-implemented method of claim 2, further comprising generating an alert in response to determining that the shipment of the at least one medication complies or does not comply with the predetermined transportation conditions.

7. The computer-implemented method of claim 6, further comprising transmitting the alert to at least one of the following: the patient, a caregiver, a pharmacy, a physician, or any combination thereof.

8. The computer-implemented method of claim 1, wherein the optimized packing configuration is determined based at least partially on an optimized packing algorithm, and wherein the optimized packing algorithm is modified and/or influenced based on the collected environmental condition data.

9. The computer-implemented method of claim 1, further comprising:
generating, with at least one processor, a unique identifier for the medication order;
storing or embedding the unique identifier in at least one data source that is affixed to a package including the medication order; and
linking, in at least one database, the unique identifier to at least a portion of the medication order.

10. The computer-implemented method of claim 9, further comprising tracking the package during shipment from the first location to the destination location by periodically scanning or reading the at least one data source to retrieve the unique identifier and transmitting, to at least one remote server, shipment data and the unique identifier.

11. The computer-implemented method of claim 1, wherein the medication order data further comprises at least one of a lot number and an expiration date, the method further comprising:
determining if the shipment should be recalled during shipment based at least partially on the at least one of the lot number and the expiration date;
generating an alert in response to determining that the shipment should be recalled; and
transmitting the alert to at least one of the following: the patient, a caregiver, a pharmacy, a physician, or any combination thereof.

12. The computer-implemented method of claim 1, further comprising:
collecting shipment data during shipment of the at least one medication from the first location to the destination location; and
after the at least one medication has been delivered to the destination location, determining whether the shipment of the at least one medication complies with manufacturer data associated with the at least one medication.

13. The computer-implemented method of claim 1, further comprising generating correlated temperature data based on past shipment data and historical environmental data, the correlated temperature data comprising correlations between ambient outdoor temperature measurements associated with past shipments and historical environmental conditions in at least one shipping lane, wherein the predicted environmental conditions are determined based at least partially on the correlated temperature data.

14. A system for predictively monitoring a shipment of at least one medication order from a first location to a destination location, comprising at least one computer including at least one processor, the at least one computer programmed or configured to:
receive a medication order comprising medication order data identifying at least one medication for a patient;
determine predicted environmental conditions for the shipment of the at least one medication from the first location to the destination location based at least partially on the medication order data;
determine an optimized packing configuration of at least one of a cooling pack and insulating material for the at least one medication order based at least partially on the predicted environmental conditions; and
generate a visual representation of the optimized packing configuration on a display device.

15. The system of claim 14, wherein the at least one computer is further programmed or configured to:
collect environmental condition data during shipment of the at least one medication from the first location to the destination location; and
determine whether the shipment of the at least one medication complies with predetermined transportation conditions by comparing the collected environmental condition data to the predetermined transportation conditions.

16. The system of claim 15, wherein the predicted environmental conditions are determined based at least partially on at least one of the following: a thermodynamic property of a packaging material, a geographical coordinate, an environmental condition, a shipping route, a mode of transportation, or any combination thereof.

17. The system of claim 16, wherein the predicted environmental conditions are determined based at least partially on at least one thermodynamic property of a packaging material.

18. The system of claim 15, wherein the predicted environmental conditions are determined based at least partially on a predictive algorithm, and wherein the predictive algorithm is modified based on the collected environmental condition data.

19. The system of claim 15, wherein the at least one computer is further programmed or configured to generate an alert in response to determining that the shipment of the at least one medication complies or does not comply with the predetermined transportation conditions.

20. The system of claim 19, wherein the at least one computer is further programmed or configured to transmit the alert to at least one of the following: the patient, a caregiver, a pharmacy, a physician, or any combination thereof.

21. The system of claim 14, wherein the optimized packing configuration is determined based at least partially on an optimized packing algorithm, and wherein the optimized packing algorithm is modified and/or influenced based on the collected environmental condition data.

22. The system of claim 14, wherein the at least one computer is further programmed or configured to:
generate a unique identifier for the medication order;
store or embed the unique identifier in at least one data source that is affixed to a package including the medication order; and
link, in at least one database, the unique identifier to at least a portion of the medication order.

23. The system of claim 22, wherein the at least one computer is further programmed or configured to track the package during shipment from the first location to the destination location by periodically scanning or reading the at least one data source to retrieve the unique identifier and transmitting, to at least one remote server, shipment data and the unique identifier.

24. The system of claim 14, wherein the medication order data further comprises at least one of a lot number and an expiration date, and wherein the at least one computer is further programmed or configured to:
determine if the shipment should be recalled during shipment based at least partially on the at least one of the lot number and the expiration date;
generate an alert in response to determining that the shipment should be recalled; and
transmit the alert to at least one of the following: the patient, a caregiver, a pharmacy, a physician, or any combination thereof.

25. The system of claim 14, wherein the at least one computer is further programmed or configured to:
collect shipment data during shipment of the at least one medication from the first location to the destination location; and
after the at least one medication has been delivered to the destination location, determine whether the shipment of the at least one medication complies with manufacturer data associated with the at least one medication.

26. The system of claim 14, wherein the at least one computer is further programmed or configured to generate correlated temperature data based on past shipment data and historical environmental data, the correlated temperature data comprising correlations between ambient outdoor temperature measurements associated with past shipments and historical environmental conditions in at least one shipping lane, wherein the predicted environmental conditions are determined based at least partially on the correlated temperature data.

27. A computer program product for predictively monitoring a shipment of at least one medication order from a first location to a destination location, comprising at least one computer-readable medium including program instructions that, when executed by at least one processor of at least one computer, cause the at least one computer to:
receive a medication order comprising medication order data identifying at least one medication for a patient;
determine predicted environmental conditions for the shipment of the at least one medication from the first location to the destination location based at least partially on the medication order data;
determine an optimized packing configuration of at least one of a cooling pack and insulating material for the at least one medication order based at least partially on the predicted environmental conditions; and
generate a visual representation of the optimized packing configuration on a display device.
